(19) United States
(12) Patent Application Publication
(10) Pub. No.: US 2024/0016867 A1
(43) Pub. Date: Jan. 18, 2024

(54) METHOD FOR PRODUCING A CANNABIS INFUSED PRODUCT

(71) Applicant: Neil McAlister, Peoria, AZ (US)

(72) Inventor: Neil McAlister, Peoria, AZ (US)

(21) Appl. No.: 17/862,682

(22) Filed: Jul. 12, 2022

(51) Int. Cl. A61K 36/185; A61K 9/00; A61K 47/26; A23L 33/105

(52) U.S. Cl. CPC A61K 36/185; A61K 9/0056; A61K 47/26; A23L 33/105; A61K 2236/333; A61K 2236/31; A23V 2002/00

(57) ABSTRACT

A method of producing a cannabis infused product that includes creating a cannabis and alcohol mixture that is filtered and frozen to create a green dragon extract that is combined with a proportional amount of sugar and baked together and dried to form the cannabis infused product. Furthermore, the instant method includes labeling the final product with information defining the medicinal qualities of the product based upon the color of the final product.

### COPYRIGHT NOTICE

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever. 37 CFR 1.71(d).

### BACKGROUND OF THE INVENTION

### 1. Field of the Invention

This invention relates generally to cannabis products, and more specifically to cannabis infused products.

### 2. Description of the Related Art

Prior art cannabis products are formed in many different varieties and methods. Many are baked directly within other food materials, such as cakes, brownies, and candies. However, there are very little controls on the quality and quantity of the cannabis and its effects on the person ingesting these products. Furthermore, cannabis, and the bi-products thereof, have differing chemical configurations and strengths and produce differing effects on the human body. To date, there has not been any known reliable method developed for knowing the exact strengths of the products derived and their exact effects on a normal person. Therefore, many people experience unwanted side effects and are not appropriately served medicinally. Accordingly, the present invention overcomes the disadvantages associated with the prior art, by providing a new and useful cannabis infused product and the method of making the cannabis infused product to thereby offer a cannabis infused that can be chosen appropriately for the desired effects necessary for that person.

### BRIEF SUMMARY OF THE INVENTION

In view of the foregoing disadvantages inherent in the known types of cannabis infused products or the like in the prior art, the present invention provides a new method of producing a cannabis infused product that includes creating a cannabis and alcohol mixture that is baked together with sugar. As such, the general purpose of the present invention, which will be described subsequently in greater detail, is to provide a method of producing a cannabis infused product that includes creating a cannabis and alcohol mixture that is filtered and frozen to create a green dragon extract that is combined with a proportional amount of sugar and baked together and dried to form the cannabis infused product. Furthermore, the instant method includes labeling the final product with information defining the medicinal qualities of the product based upon the color of the final product, with all the advantages of the prior art and none of the disadvantages.

There has thus been outlined, rather broadly, the more important features of the invention in order that the detailed description thereof that follows may be better understood and in order that the present contribution to the art may be better appreciated.

Numerous objects, features and advantages of the present invention will be readily apparent to those of ordinary skill in the art upon a reading of the following detailed description of presently preferred, but nonetheless illustrative, embodiments of the present invention when taken in conjunction with the accompanying drawings. The invention is capable of other embodiments and of being practiced and carried out in various ways. Also, it is to be understood that the phraseology and terminology employed herein are for the purpose of descriptions and should not be regarded as limiting.

### BRIEF DESCRIPTION OF THE DRAWINGS

The figures which accompany the written portion of this specification illustrate embodiments according to the teachings of the present invention.

FIG. 1 shows a flowchart of the method of producing the cannabis infused product according to the preferred embodiment of the present invention.

FIG. 2 shows a view of the material elements of the method of producing the cannabis infused product according to the preferred embodiment of the present invention of FIG. 1.

FIG. 3 shows a view of vials of the cannabis infused product sorted by color representing medicinal qualities of the product within each vial according to the preferred embodiment of the present invention of FIG. 1.

The various embodiments of the present invention will hereinafter be described in conjunction with the appended drawings.

### DETAILED DESCRIPTION

The embodiments of the present disclosure described below are not intended to be exhaustive or to limit the disclosure to the precise forms disclosed in the following detailed description. Rather, the embodiments are chosen and described so that others skilled in the art may appreciate and understand the principles and practices of the present disclosure.

The following embodiments and the accompanying drawings, which are incorporated into and form part of this disclosure, illustrate embodiments of the invention and together with the description, serve to explain the principles of the invention. To the accomplishment of the foregoing and related ends, certain illustrative aspects of the invention are described herein in connection with the following description and the annexed drawings. These aspects are indicative, however, of but a few of the various ways in which the principles of the invention can be employed and the subject invention is intended to include all such aspects and their equivalents. Other advantages and novel features of the invention will become apparent from the following detailed description of the invention when considered in conjunction with the drawings.

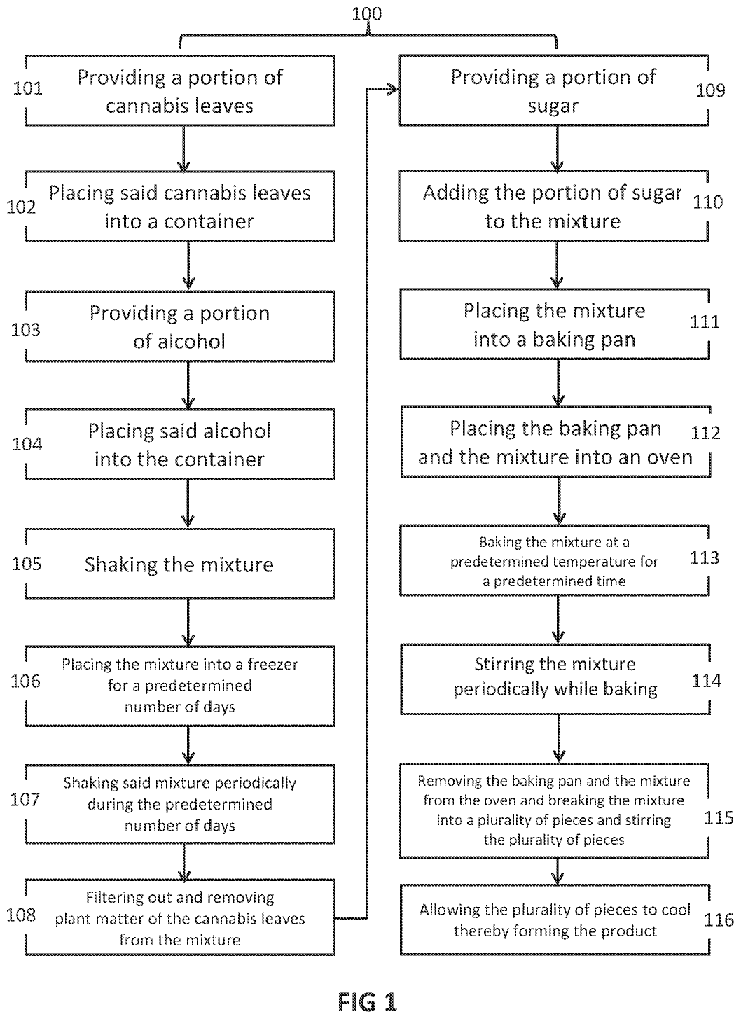

Turning now to FIGS. 1-3, the present invention discloses a method of producing a cannabis infused product 100 comprising the steps of providing a portion of cannabis leaves 101; placing the cannabis leaves into a container 102; providing a portion of alcohol 103; placing the alcohol into the container 104, wherein the portion of the cannabis and the portion of the alcohol produces a mixture; shaking the mixture 105; placing the mixture into a freezer for a predetermined number of days 106; shaking the mixture periodically during the predetermined number of days 107; filtering out and removing plant matter of the cannabis leaves from the mixture 108; providing a portion of sugar 109; adding the portion of sugar to the mixture 110; placing the mixture into a baking pan 111; placing the baking pan and the mixture into an oven 112; baking the mixture at a predetermined temperature for a predetermined time 113; stirring the mixture periodically while baking 114; removing the baking pan and the mixture from the oven and breaking the mixture into a plurality of pieces and stirring the plurality of pieces 115; and then allowing the plurality of pieces to cool thereby forming the product 116.

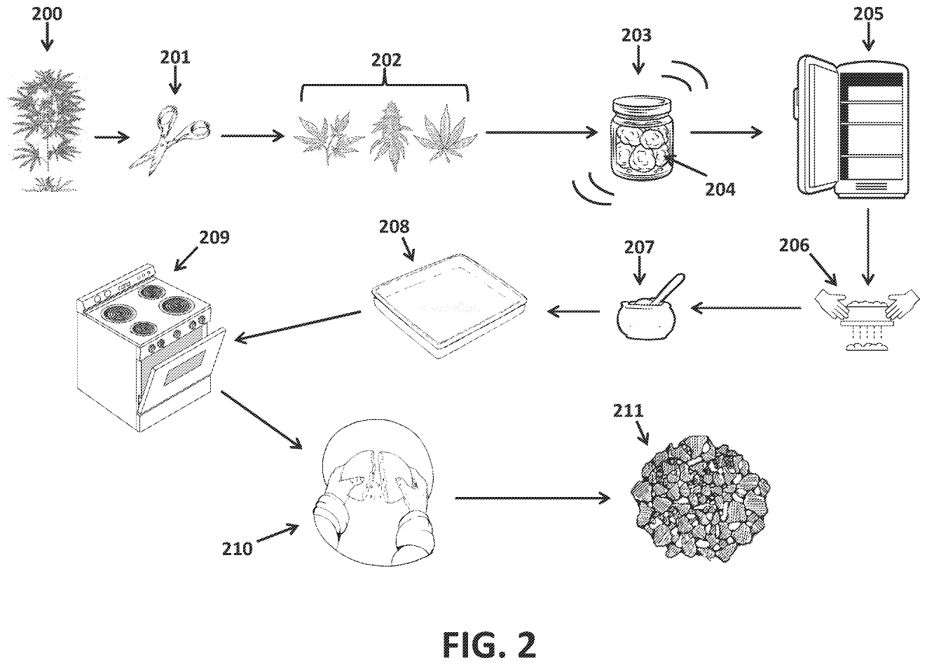

As shown in FIG. 2, the cannabis plant 200 is cut using a cutting tool 201 to retrieve either leaves, which can also be trim or buds 202; placing the cannabis leaves into a container 203 and including a portion of alcohol 204 forming a mixture, and shaking the mixture; placing the mixture into a freezer 205 for a predetermined number of days; filtering out and removing plant matter of the cannabis leaves from the mixture using a filter 206 or the like; adding a portion of sugar 207 to the mixture; placing the mixture into a baking pan 208; placing the baking pan and the mixture into an oven 209; baking the mixture at a predetermined temperature for a predetermined time and stirring the mixture periodically while baking; removing the baking pan and the mixture from the oven and breaking the mixture into a plurality of pieces 210 and stirring the plurality of pieces and allowing the plurality of pieces to cool thereby forming the product 211.

In the preferred embodiment, the step of placing the mixture into a freezer is for seven days; the step of filtering out and removing plant matter is performed a plurality of times in succession; the baking pan is formed from glass; the step of baking the mixture is at 170 degrees Fahrenheit for one hour; the step of stirring the mixture periodically while baking is performed at intervals of 15 minutes; the step of shaking the mixture periodically during the predetermined number of days comprises shaking the mixture three times per day for one minute intervals each time; and the step of providing a portion of cannabis leaves includes harvesting the cannabis leaves from its respective cannabis plant and placing the cannabis leaves into the container within a time period of 12 to 24 hours from the harvesting. Furthermore, in the preferred embodiment the proportion of the sugar to the mixture is three parts sugar to one part mixture, wherein the portion of sugar has a volume of 1 cup and wherein the mixture has a volume of ⅓ of a cup. It should be noted that ⅓ of a cup of alcohol weighs 65.37 grams, and 1 cup of sugar weighs 200 grams.

The portion of the cannabis plant that is cut and used within the instant method and process can be called “Sugarleaf”. The mixture created when combining the cannabis leaf with alcohol can be referred to as “Green Dragon”. When discussing the term cannabis leaf you can use either the cannabis flower meaning the buds or the trim to get the desired results. For the highest quality product, the “Sugarleaf” has to be used within 24 hours of harvesting and putting it straight into the extraction to start that process.

The alcohol used within the instant method can be alcohol of differing strengths. In the preferred embodiment, grain alcohol is used having a 90% alcohol by composition, which correlates to 180 proof.

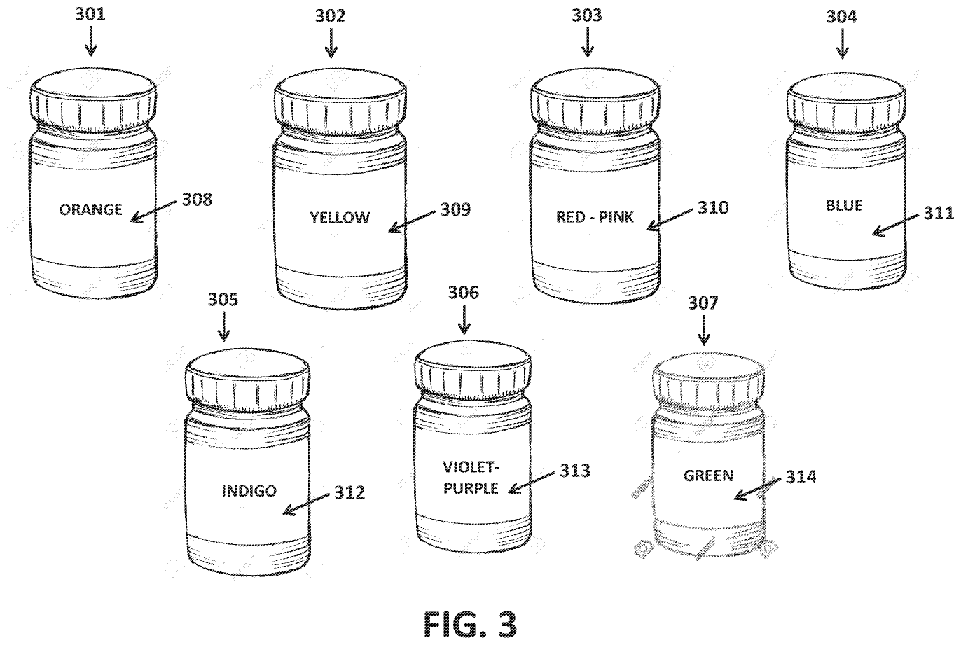

The cannabis leaves may be cultivated from either the cannabis flowers or from cannabis trim. Each cannabis plant will produce a product varying strength and varying medicinal qualities. Furthermore, each part of the cannabis plant will produce a different final product having varying strength and varying medicinal qualities as well. The varying chemical configurations of different cannabis plants produces varying colors of the final cannabis infused product that actually correlate to particular medicinal qualities of that final cannabis infused product. As such, and as shown in FIG. 3, the instant method of producing the cannabis infused product further includes the steps of placing the product into secondary containers 301-307; providing a label 308-314 upon the secondary containers providing printed information upon the label setting forth medicinal information of the product based on its color. The medicinal information of the product based on its color includes:
ORANGE—for mood enhancing and stress relief;
YELLOW—sharpens creativity, increases sensory awareness, increases happiness, and provides uplifting effects;
RED or PINK—aids in pain relief, insomnia, and increases appetite;
BLUE—reduces muscle spasms, provides pain relief, reduces stress, and increases relaxation;
INDIGO—increases cerebral highs, increases and enhances psychedelic affects;
VIOLET/PURPLE—increases pain relief, and reduces insomnia; and
GREEN—reduces fatigue, stress, and depression.

Although specific embodiments have been illustrated and described herein, it will be appreciated by those of ordinary skill in the art that any arrangement, which is calculated to achieve the same purpose, may be substituted for the specific embodiment shown. This application is intended to cover any adaptations or variations of the present invention.

Although the invention has been explained in relation to its preferred embodiment, it is to be understood that many other possible modifications and variations can be made without departing from the spirit and scope of the invention.

What is claimed is:

1. A method of producing a cannabis infused product comprising the steps of:
providing a portion of cannabis leaves;
placing said cannabis leaves into a container;
providing a portion of alcohol;
placing said alcohol into said container;
wherein said portion of said cannabis and said portion of said alcohol produces a mixture;
shaking said mixture;
placing said mixture into a freezer for a predetermined number of days;
shaking said mixture periodically during said predetermined number of days;
filtering out and removing plant matter of said cannabis leaves from said mixture;
providing a portion of sugar;
adding said portion of sugar to said mixture;
placing said mixture into a baking pan;
placing said baking pan and said mixture into an oven;
baking said mixture at a predetermined temperature for a predetermined time;
stirring said mixture periodically while baking;
removing said baking pan and said mixture from said oven;
breaking said mixture into a plurality of pieces and stirring said plurality of pieces; and
allowing said plurality of pieces to cool thereby forming said product.

2. The method of producing a cannabis infused product of claim 1, wherein said step of placing said mixture into a freezer is for seven days.

3. The method of producing a cannabis infused product of claim 1, wherein said step of filtering out and removing plant matter is performed a plurality of times in succession.

4. The method of producing a cannabis infused product of claim 1, wherein said baking pan of said step of placing said mixture into a baking pan is formed from glass.

5. The method of producing a cannabis infused product of claim 1, wherein said step of baking said mixture is at 170 degrees Fahrenheit for one hour.

6. The method of producing a cannabis infused product of claim 1, wherein said step of stirring said mixture periodically while baking is performed at intervals of 15 minutes.

7. The method of producing a cannabis infused product of claim 1, wherein said step of shaking said mixture periodically during said predetermined number of days comprises shaking said mixture three times per day for one minute intervals each time.

8. The method of producing a cannabis infused product of claim 1, wherein said step of providing a portion of cannabis leaves includes harvesting said cannabis leaves from its respective cannabis plant and placing said cannabis leaves into said container within a time period of 12 to 24 hours from said harvesting.

9. The method of producing a cannabis infused product of claim 1, wherein the proportion of said sugar to said mixture is three parts sugar to one part mixture.

10. The method of producing a cannabis infused product of claim 9, wherein said portion of sugar has a volume of 1 cup; and wherein said mixture has a volume of ⅓ of a cup.

11. The method of producing a cannabis infused product of claim 1, wherein said cannabis leaves are cultivated from cannabis flowers.

12. The method of producing a cannabis infused product of claim 1, wherein said cannabis leaves are cultivated from cannabis trim.

13. The method of producing a cannabis infused product of claim 1, further including the step of:
placing said product into a secondary container;
providing a label upon said secondary container;
providing printed information upon said label;
wherein said printed information sets forth medicinal information of said product based on its color.

14. The method of producing a cannabis infused product of claim 13, wherein said medicinal information of said product based on its color includes choosing from a list of medicinal information of said product based on its color comprising:
orange—for mood enhancing and stress relief;
yellow—sharpens creativity, increases sensory awareness, increases happiness, and provides uplifting effects;
red or pink—aids in pain relief, insomnia, and increases appetite;
blue—reduces muscle spasms, provides pain relief, reduces stress, and increases relaxation;
indigo—increases cerebral highs, increases and enhances psychedelic affects;
violet/purple—increases pain relief, and reduces insomnia; and
green—reduces fatigue, stress, and depression.
